(19) United States
(12) Patent Application Publication
(10) Pub. No.: US 2011/0306912 A1
(43) Pub. Date: Dec. 15, 2011

(54) Multi-purpose Toe Strips

(76) Inventor: Pamela Funderburg Heckel, Wyoming, OH (US)

(21) Appl. No.: 13/157,358

(22) Filed: Jun. 10, 2011

Related U.S. Application Data
(60) Provisional application No. 61/353,919, filed on Jun. 11, 2010.

(51) Int. Cl. A61F 5/00

(52) U.S. Cl. 602/30

(57) ABSTRACT

The present invention may be used to provide relief from pain due to misaligned toes and may also be used to deliver pharmacological agents between the toes to treat diseases of the foot. The invention may consist of a single strip of a “sponge” fabric that is woven between the toes and fastened with a water-soluble adhesive to the top of the foot.

### CROSS REFERENCE TO RELATE APPLICATION

Provisional patent application 61353919 filed Jun. 11, 2010, titled Multi-purpose Toe Strips

### BACKGROUND

This invention relates to an apparatus for the mechanical treatment of several ailments of the foot, including hammertoes, overlapping toes, and pinched toes. Hammertoes are caused by a muscle imbalance that makes the ligaments and tendons of the foot unnaturally tight. The result of the tight ligaments is a toe joint that curls downward. The top of the toes may rub against the inside of the shoe. Overlapping toes are when the toes rest on top of each other. This can cause discomfort due to friction, especially when the top toe is in contact with the inside top surface of the shoe. Pinched toes mash and rub against each other causing corns to develop and may be painful. The invention may be used to reposition the dominant toe.

This invention may also be used to deliver medications between the toes. Athlete's foot is a fungal infection. The best medicine currently on the market guarantees the healing of skin on the outside of the foot, but specifically excludes the areas between the toes. The invention may address that limitation by being a delivery agent for medicines intended to treat bacterial/fungal infections between the toes.

Various prosthetic devices to treat foot ailments may be known. However, the distinguishing characteristics of this invention mechanically address the described foot ailments without the known medical complications associated with other prosthetic devices such as: difficulty of application, constriction of blood flow through the toes and secondary infections.

### SUMMARY OF THE INVENTION The Multi-purpose Toe Strip comprises a single part made of absorbent material, a “sponge”. The device may be woven between the toes on a human foot and attached to the top of the foot. The device may be used for the physical realignment of the toes and/or delivery of chemical/pharmaceutical agents.

Versions can be used to hold toes apart from each other, and to deliver medications or absorb moisture between the toes. Embodiments wrap around the foot and between the toes starting with the widest portion on the top of the foot above the largest toe and proceeding around and between the toes to end with the smaller end on top of the foot above the smallest toe. Versions can deliver medications to the outside of the big and little toes. A light coating of water-soluble adhesive may be applied to the ends of the invention to attach those ends to the top of the foot.

These and other features, aspects and advantages of the present invention will become better understood with reference to the following drawings, description and claims.

### BRIEF DESCRIPTION OF DRAWINGS

FIG. 1 illustrates the top and longitudinal side views of the invention.

FIG. 2 illustrates the weaving of the strip between the toes

FIG. 3 illustrates the strip woven between the toes and attached to the top of the foot

### DETAILED DESCRIPTION

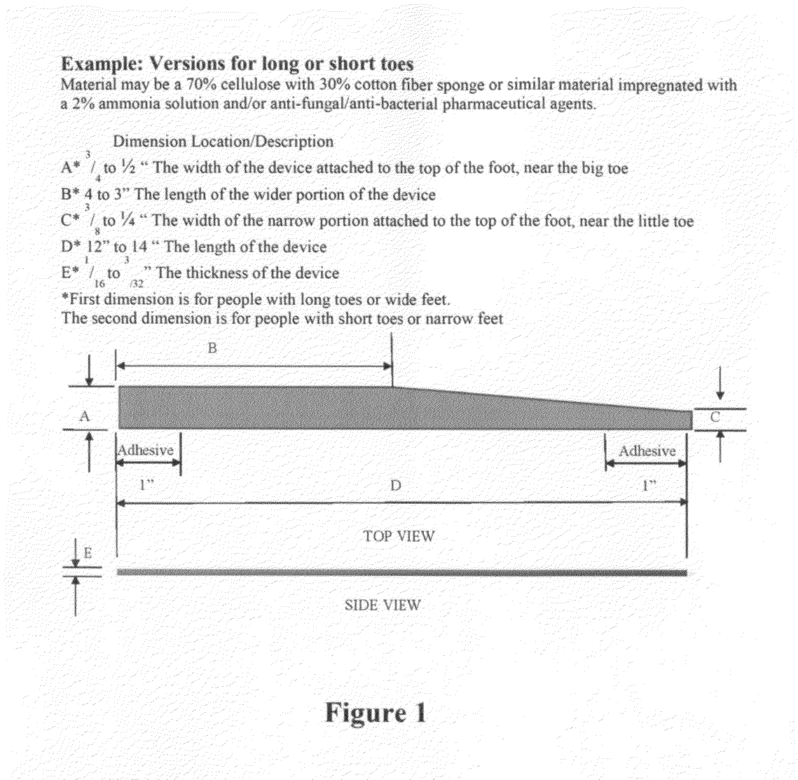

The invention comprises a single part made of absorbent material, a “sponge”, which may have a trapezoidal shape as shown in FIG. 1. The invention is a multi-functional device which can be used to deliver medicine to the toes and foot and may be worn as a prosthetic device to address the condition of misaligned toes. When worn with properly fitted footwear, the MULTI-PURPOSE TOE STRIPS device may relieve: Hammertoes, Overlapping toes, and Pinched toes. The invention may be impregnated with anti-fungal and/or anti-bacterial medications to assist in the treatment of infections between the toes.

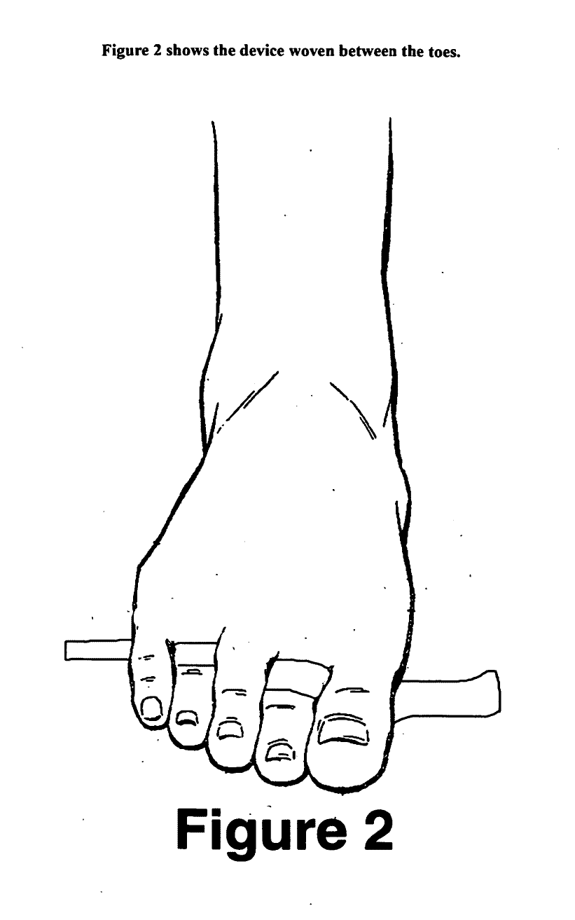

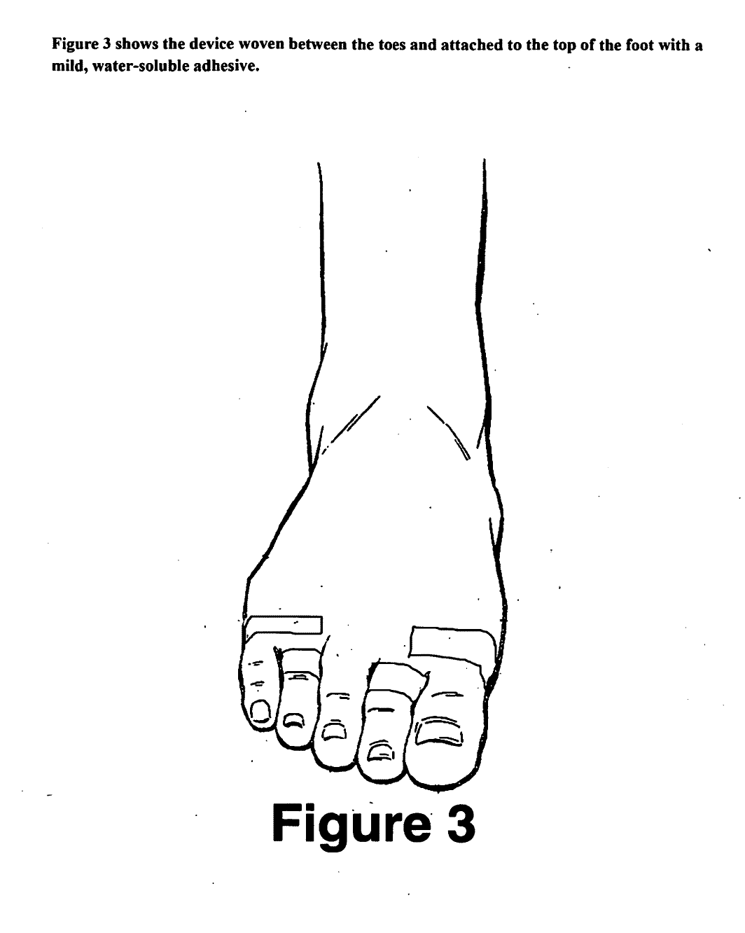

Versions can be used to address misalignment of the toes by repositioning the dominant toe. As shown in FIGS. 2 and 3, the invention wraps around the foot and between the toes starting with the widest portion of the strip on the top of the foot above the largest toe and proceeding around and between the toes to end with the smaller end on top of the foot above the smallest toe. The soft sponge is woven between toes. In one version, the invention covers the part of the toe closest to the foot, does not extend beyond the first joint of the second toe, and is not as wide as the smallest toe is long. The invention separates and realigns the toes as it wraps under the big toe, over the second toe, under the third toe, over the fourth toe, and finally under the smallest toe. The ends wrap around the outside of the largest and smallest toes and attach to the top of the foot with water soluble adhesive on each end. The length of the individual toe cover varies with the length of the toes. Similarly, the shape of the strip may be configured to accommodate the difference in toe length between the big toe and the smallest toe. In one version, the trapezoidal shape fits between the toes from the base of the toe valley up to the first toe knuckle.

The invention fits loosely between the toes, so it does not restrict blood circulation. The invention distributes the pressure across each toe with each step, gently pulling the misaligned toes back into alignment.

The invention comprises a “sponge” which can contain natural and synthetic materials. In one version, the invention is a “sponge” with a water-soluble adhesive applied at each end of the “sponge”. The water-soluble adhesive on the invention weakly binds the sponge to the top of the foot yet it holds the device in place while a sock or stocking is donned. When it is removed, it does not pull the hair. The invention may be a single piece of material.

The invention may be an agent to deliver liquid or cream medications, including anti-bacterial/anti-fungal medications.

Versions may or may not completely encircle and separate each toe for all the toes on the foot. The invention may be woven over-under the toes. The ends of the invention may be glued to the foot. The invention may permit normal toe movement. The portion of the sponge over the second toe protects a misaligned toe from rubbing against the inside top surface of the shoe. The portions of the invention that extend under the big toe and under the third toe respond to the pressure created during walking to realign all three affected toes. The tapered width of the invention, in one version, is designed to extend from the valley between the toes to the first knuckle on the toe. The wider end is intended for the big toe and the smaller end is intended for the little toe. The invention may be designed to accommodate the normal gripping action of the toes.

When the invention has been particularly shown and described with respect to the illustrated embodiments thereof, it will be understood by those skilled in the art that the foregoing and other changes in form and details may be made therein without departing from the spirit and scope of the invention.

i. The invention rests lightly on the foot, under the socks or stockings, and does not interfere with the fit of footwear.

ii. The invention is a single piece of hydrophilic material that may demonstrate an ability to absorb moisture between the toes.

iii. The invention gently realigns the toes but does not enclose or bind them.

iv. The invention is attached to the top of the foot by means of a water-soluble adhesive that weakly binds the invention to the foot yet it holds the device in place while a sock or stocking is donned. When the invention is removed from the foot, it does not pull the hair or damage the skin.

v. The invention may be used to topically deliver chemical/pharmacological agents to the skin between the toes to treat bacterial/fungal infections.

vi. The invention is made of a “sponge” which can contain natural and synthetic materials. The fibers of the “sponge” may be aligned perpendicular to the toe to provide greater efficacy for the correction of the misaligned toes.

vii. The invention is flexible, hydrophilic, resistant to mold, fungus and mildew and is intended to be disposable.
